(19) United States
(12) Patent Application Publication
(10) Pub. No.: US 2018/0296827 A1
(43) Pub. Date: Oct. 18, 2018

(54) DIRECTIONAL ELECTRICAL STIMULATION LEADS AND SYSTEMS WITH ANCHORING UNITS AND METHODS OF MAKING AND USING

(71) Applicant: Boston Scientific Neuromodulation Corporation, Valencia, CA (US)

(72) Inventors: Anne Margaret Pianca, Santa Monica, CA (US); Rafael Carbunaru, Valley Village, CA (US)

(21) Appl. No.: 15/945,100

(22) Filed: Apr. 4, 2018

Related U.S. Application Data
(60) Provisional application No. 62/484,844, filed on Apr. 12, 2017.

(51) Int. Cl. A61N 1/05; A61N 1/36

(52) U.S. Cl. CPC A61N 1/0558; A61N 1/36007; A61N 1/36128; A61N 1/36071; A61N 1/36107; A61N 1/36062

(57) ABSTRACT

An electrical-stimulation lead includes a body having a distal portion, a proximal portion, and a perimeter; electrodes disposed along the distal portion of the body and including segmented electrodes that each extend around no more than 50% of the perimeter of the body; terminals disposed along the proximal portion of the body; and conductors electrically-coupling the terminals to the electrodes; and at least one anchoring unit disposed along the distal portion of the body and configured and arranged for resisting rotation of the body and electrodes relative to patient tissue upon implantation of the lead. Each anchoring unit includes a lead-attachment element and at least one anchoring element attached to the lead-attachment element. The anchoring element extends away from the lead-attachment element and is configured and arranged for physically contacting patient tissue and resisting rotation of the body in proximity to the anchoring element.

### CROSS-REFERENCE TO RELATED APPLICATIONS

This application claims the benefit under 35 U.S.C. § 119(e) of U.S. Provisional Patent Application Ser. No. 62/484,844, filed Apr. 12, 2017, which is incorporated herein by reference.

### FIELD

The present invention is directed to the area of implantable electrical stimulation systems and methods of making and using the systems, and in particular implantable electrical stimulation leads having anchoring units and methods of making and using the leads.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

One concern regarding implanted leads (or control modules) is migration. This may occur over time and result in movement of the stimulator electrodes away from the desired tissue for stimulation so as to reduce the effectiveness of therapeutic treatment.

### BRIEF SUMMARY

One embodiment is an implantable electrical stimulation lead that includes a lead body having a distal portion, a proximal portion, a perimeter, and a longitudinal length; electrodes disposed along the distal portion of the lead body and including at segmented electrodes that each extend around no more than 50% of the perimeter of the lead body; terminals disposed along the proximal portion of the lead body; and conductors electrically coupling the terminals to the electrodes; and at least one anchoring unit disposed along the distal portion of the lead body and configured and arranged for resisting rotation of the lead body and electrodes relative to patient tissue after implantation of the lead. Each anchoring unit includes a lead attachment element and at least one anchoring element attached to the lead attachment element. The lead attachment element defines a central lumen within which a portion of the lead body is received and has a proximal end, a distal end, a diameter, and a longitudinal length. The anchoring element extends away from the lead attachment element and is configured and arranged for physically contacting patient tissue and resisting rotation of the lead body in proximity to the anchoring element. In at least some embodiments, the anchoring unit is permanently attached to the lead body or molded with the lead body. In at least some embodiments, the anchoring unit is placed or positioned on the lead body after manufacture.

In at least some embodiments, the at least one anchoring element is attached to the lead attachment element at the proximal end of the lead attachment element. In at least some embodiments, the at least one anchoring element is attached to the lead attachment element at the distal end of the lead attachment element. In at least some embodiments, the at least one anchoring element is attached to the lead attachment element exclusively at an intermediate portion of the lead attachment element between the proximal end and the distal end.

In at least some embodiments, each anchoring element includes a cone having a first end and an opposing second end, where the first end is attached to the lead attachment element and the second end is longitudinally spaced away from the lead attachment element.

In at least some embodiments, each anchoring element includes at least one foot disposed along the second end of the cone. In at least some embodiments, each anchoring element includes fins, each fin attached to, and extending away from, the lead attachment element. In at least some embodiments, each of the fins defines a cutout.

In at least some embodiments, each anchoring element includes a first member attached to the lead attachment element at a first location and a second member attached to the lead attachment element at a second location that is discrete from, and proximally-offset from, the first location along the longitudinal length of the lead attachment element. In at least some embodiments, the first member extends from the lead attachment element and couples to the second member to form at least one aperture bounded, at least in part, by the first member and the second member. In at least some embodiments, the first member extends along an arced path from the second member to the lead attachment element. In at least some embodiments, the first member has a width that tapers from the second member to the lead attachment element. In at least some embodiments, the second member extends from the lead attachment element at the proximal end of the lead attachment element. In at least some embodiments, the second member extends from the lead attachment element at a 90-degree angle from the lead attachment element. In at least some embodiments, the second member extends from the lead attachment element at an angle, in a range from 30 to 90 degrees, from the lead attachment element.

In at least some embodiments, each anchoring element extends away from the lead attachment element by an amount that is greater than the longitudinal length of the lead attachment element.

Another embodiment is an electrical stimulation system that includes the electrical stimulation lead described above and a control module coupleable to the electrical stimulation lead.

Yet another embodiment is a method for sacral stimulation to treat a condition. The method includes advancing the distal portion of any one of the electrical stimulation leads described above to a location within a patient in proximity to at least one nerve root of at least one of the S1, S2 S3, S4, or S5 level of the patient's spinal cord; anchoring the distal portion of the electrical stimulation lead to patient tissue using the anchoring unit to resist rotational movement of the electrodes of the electrical stimulation lead relative to the at least one nerve root; and providing electrical signals from a control module coupled to the electrical stimulation lead to stimulate the at least one nerve root using the electrodes of the electrical stimulation lead.

In at least some embodiments, anchoring the distal portion of the electrical stimulation lead to patient tissue includes anchoring the distal portion of the electrical stimulation lead to patient tissue to resist axial movement of the plurality of electrodes of the electrical stimulation lead relative to the at least one nerve root. In at least some embodiments, the condition is at least one of overactive bladder, urinary incontinence, bowel incontinence, urinary retention, fecal retention, fecal incontinence, intersticial cistytis, pelvic pain, erectile dysfunction, female sexual dysfunction, or pelvic floor tone dysfunctions.

Still another embodiment is an implantable microstimulator that includes a control module with a housing having an outer surface and an electronic subassembly disposed in the housing; electrodes disposed along the outer surface of the housing and coupled to the electronic subassembly; and at least one anchoring unit disposed along the outer surface of the control module and configured and arranged for resisting movement of the control module relative to patient tissue after implantation of the microstimulator. The anchoring unit includes a lead attachment element and at least one anchoring element. The lead attachment defines a central lumen within which a portion of the control module is received and has a proximal end, a distal end, a diameter, and a longitudinal length. The anchoring element is attached to the lead attachment element and extends away from the lead attachment element. The anchoring element is configured and arranged for physically contacting patient tissue and resisting movement of the control module in proximity to the at least one anchoring element.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIGS. 2A-2D are schematic side views of four alternate embodiments of distal portions of an electrical stimulation lead, according to the invention;

FIG. 3 is a schematic perspective view of one embodiment of a lead anchoring unit, according to the invention;

FIG. 4 is a schematic perspective view of a second embodiment of a lead anchoring unit, according to the invention;

FIG. 5 is a schematic perspective view of a third embodiment of a lead anchoring unit, according to the invention;

FIG. 6 is a schematic perspective view of a fourth embodiment of a lead anchoring unit, according to the invention;

FIG. 7 is a schematic perspective view of a fifth embodiment of a lead anchoring unit, according to the invention;

FIG. 8 is a schematic perspective view of a sixth embodiment of a lead anchoring unit, according to the invention;

FIG. 9 is a schematic perspective view of a seventh embodiment of a lead anchoring unit, according to the invention; and

FIGS. 10A-10F are schematic side views of several different alternate embodiments of a distal portion of a lead with lead anchoring units disposed thereon, according to the invention.

### DETAILED DESCRIPTION

The present invention is directed to the area of implantable electrical stimulation systems and methods of making and using the systems, and in particular implantable electrical stimulation leads having anchoring units and methods of making and using the leads.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference.

Suitable implantable electrical stimulation systems may also include one or more microstimulators, which include an implantable control module containing electrical circuitry connected to one or more electrodes that extend through, or along, one or more walls of the control module. In some instances, microstimulators include segmented electrodes. Examples of microstimulators are found in, for example, U.S. Pat. Nos. 5,193,539; 5,193,540; 5,312,439; 5,324,316; 5,405,367; 7,660,631; 8,214,048; 9,283,394; and U.S. Patent Applications Publication No. 2006/0036286, all of which are incorporated by reference.

In the discussion below, a percutaneous lead will be exemplified, but it will be understood that the methods and systems described herein are also applicable to paddle leads and other leads, as well as to microstimulators.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

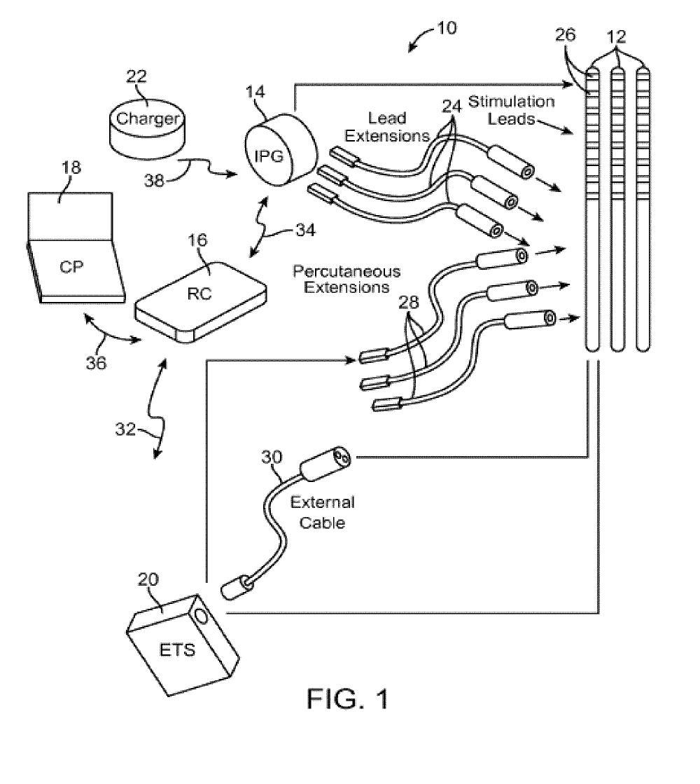

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

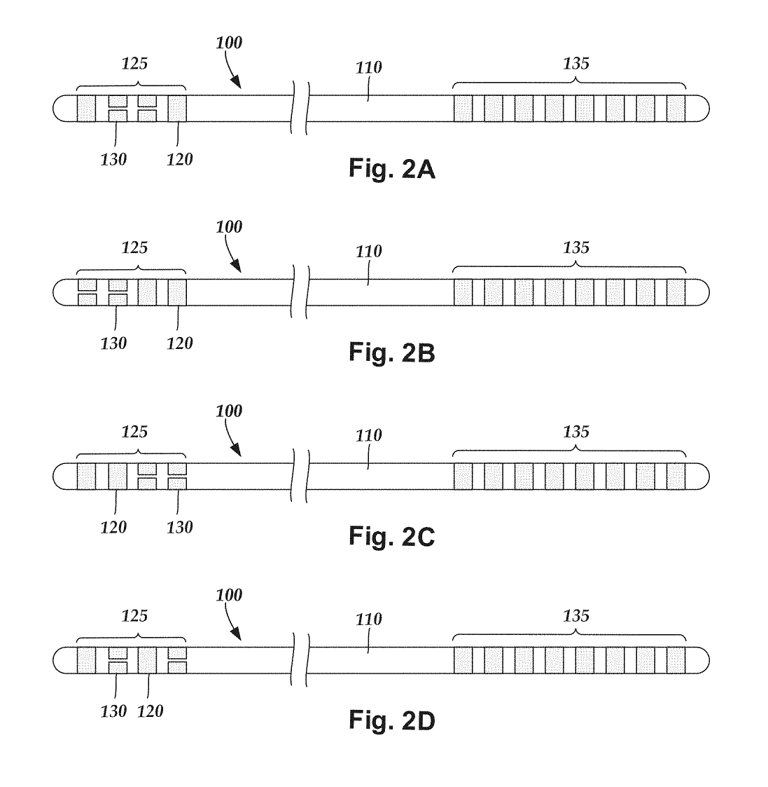

FIGS. 2A-2D illustrate several exemplary embodiments of a lead 110 with electrodes 125 disposed at least partially about a circumference of the lead 110 along a distal end portion of the lead and terminals 135 disposed along a proximal end portion of the lead. The lead 110 can be implanted near or within the desired portion of the body to be stimulated such as, for example, the brain, spinal cord, or other body organs or tissues.

In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 110 can be inserted into the cranium and brain tissue with the assistance of a stylet (not shown). The lead 110 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 110, advance the lead 110, retract the lead 110, or rotate the lead 110.

In another example of operation for sacral nerve stimulation, access to the desired position in proximity to one or more portions of the sacrum, such as one or more nerve roots of one or more of the S1, S2, S3, S4, or S5 level of the spinal cord, can be accomplished by extending the lead through one of the sacral foramen. A stylet may be used to assist advancement of the lead through a needle or introducer that has been extended through the foramen and positioned with a distal end of the needle or introducer positioned in proximity to the target stimulation location.

Electrodes may be disposed on the circumference of the lead 110 to stimulate the target tissue. Stimulation electrodes may be ring-shaped so that current projects from each electrode equally in every direction from the position of the electrode along a length of the lead 110. In the embodiment of FIG. 2, two of the electrodes 125 are ring electrodes 120. Ring electrodes typically do not enable stimulus current to be directed from only a limited angular range around of the lead. Segmented electrodes 130, however, can be used to direct stimulus current to a selected angular range around the lead. When segmented electrodes are used in conjunction with an implantable pulse generator that delivers constant current stimulus, current steering can be achieved to more precisely deliver the stimulus to a position around an axis of the lead (i.e., radial positioning around the axis of the lead). To achieve current steering, segmented electrodes can be utilized in addition to, or as an alternative to, ring electrodes.

The lead 100 includes a lead body 110, terminals 135, and one or more ring electrodes 120 and one or more sets of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use.

Each of the electrodes can either be used or unused (OFF). When the electrode is used, the electrode can be used as an anode or cathode and carry anodic or cathodic current. In some instances, an electrode might be an anode for a period of time and a cathode for a period of time.

Segmented electrodes may provide for superior current steering than ring electrodes because target structures may not be symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; and 8,792,993; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; 2015/0051681; U.S. patent applications Ser. Nos. 14/557,211 and 14/286,797; and U.S. Provisional Patent Application Ser. No. 62/113,291, all of which are incorporated herein by reference. Segmented electrodes can also be used for other stimulation techniques including, but not limited to, spinal cord stimulation, peripheral nerve stimulation, dorsal root ganglion stimulation, or stimulation of other nerves, muscles, and tissues.

The relative positioning of the ring electrodes 120 to the segmented electrodes 130 can be varied. For example, FIG. 2A shows the electrodes arranged with two sets of segmented electrodes 130 being flanked both proximally and distally by ring electrodes 120. A naming nomenclature can be used as a shorthand way to describe the various potential electrode arrangements. In the case where the segmented electrodes are arranged in sets of three electrodes disposed around a circumference of the lead, the electrode arrangement shown in FIG. 2A can be written as a 1-3-3-1 arrangement, where the numbers correspond to the number of electrodes circumferentially distributed about the lead at a particular longitudinal position proceeding proximally from the distal tip. Using such a numbering nomenclature for FIGS. 2B-2D shows a 3-3-1-1 arrangement in FIG. 2B; a 1-1-3-3 arrangement in FIG. 2C; and a 1-3-1-3 arranged in FIG. 2D. Other combinations are possible, for example, 3-1-1-3 and 3-1-3-1. Note that these examples are all based on an 8-electrode arrangement with the segmented electrodes all being formed in sets of three. Other combinations are possible with segmented electrodes being formed of sets with different numbers of electrodes than three and/or electrode arrangements having a different number of electrodes than eight.

As discussed above, directional stimulation enables improved selectivity of stimulation, as compared stimulation exclusively by ring electrodes. Improved selectivity of stimulation can reduce undesired collateral stimulation of tissue at, or in proximity to, a target stimulation region. For example, it may be desirable to direct stimulation to one or more target nerve roots (e.g., S1, S2, S3, S4, S5, within sacrum and peripheral nerves, the cauda equina of spinal cord from L1-L5, or a combination thereof) while avoiding stimulation of muscle tissue in close proximity to those one or more nerve roots.

In the case of sacral nerve stimulation, when using non-directional leads there can be side effects including, for example, undesired motor stimulation, pain, adverse change in bowel function, and adverse change in voiding function. Directional electrodes would provide with more options for “tuning” the therapy to reduce, or even eliminate, these side effects.

Furthermore, each nerve contains multiple fascicles that carry different information. With directional electrodes, one can more precisely target fascicle fibers within the nerve. Exactly what signals are carried within each fascicle is not currently well understood. It may be the case that some fascicles might carry signals that are beneficial for treating overactive bladder, whereas others might carry signals that are beneficial for treating fecal incontinence. The opposite can be the case in certain fascicles (i.e., certain fascicles carry signals that are not beneficial for treating a given condition). With a directional lead, targeting fascicles beneficial to a given treatment, while avoiding nonbeneficial fascicles may be possible. Additionally or alternatively, directional electrodes can also be used to select branches of nerves in the sacrum, epidural space or periphery.

Additionally, directional stimulation may enable concurrent or sequential targeted stimulation at multiple stimulation locations from a single implantation site. Thus, therapy may be provided for multiple indications (e.g., overactive bladder, urinary incontinence, bowel incontinence, urinary retention, fecal retention, fecal retention, fecal incontinence, intersticial cistytis, pelvic pain, erectile dysfunction, female sexual dysfunction, or pelvic floor tone dysfunctions, or the like) from a single implantation site without undesirably stimulating all excitable tissue in proximity to that implantation site.

The targets for stimulation for treating one or more of overactive bladder, urinary incontinence, bowel incontinence, urinary retention, fecal retention fecal retention, fecal incontinence, intersticial cistytis, pelvic pain, erectile dysfunction, female sexual dysfunction, or pelvic floor tone dysfunctions, include sacral nerve stimulation, peripheral nerve stimulation, tibial nerve stimulation, pudendal nerve stimulation, dorsal genital stimulation, paraurethral stimulation, clitoral nerve stimulation, sciatic nerve stimulation, genitofemoral nerve stimulation, vaginal stimulation, dorsal genital nerve stimulation, as well as subcutaneous stimulation of the pelvic region, sacral plexus, coccygeal plexus, and the cauda equina of spinal cord from L1-L5. It has been observed that patients with overactive bladder often develop bowel, or fecal, incontinence over time, and stimulation in the same location as is used for overactive bladder can also lessen the symptoms of fecal incontinence. Of the targets mentioned, sacral nerve stimulation may be the most effective.

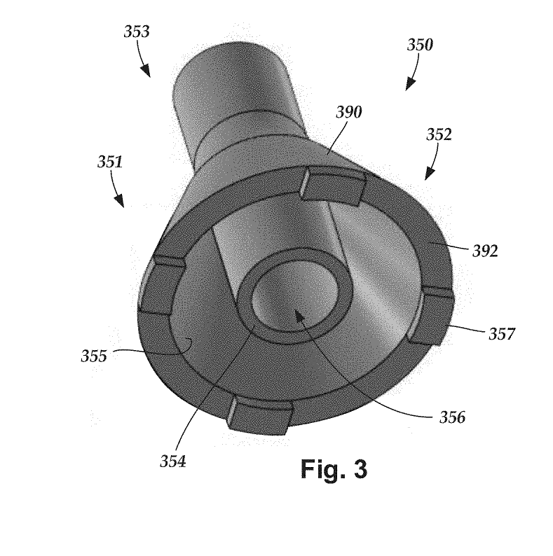

Turning to FIGS. 3-10F, using one or more lead anchoring units may be advantageous for maintaining the positioning of the lead while stimulating tissue. Lead anchoring units can be attached to the lead to facilitate anchoring the lead into patient tissue. The term “tissue” includes, but is not limited to, muscular tissue, connective tissue, organ tissue, bone, cartilage, nerve tissue, adipose tissue, and the like. These lead anchoring units, as opposed to conventional lead anchors, can be delivered with the lead through a needle or introducer during the implantation process. In some embodiments, the lead anchoring units maintain a low-profile configuration against a surface of the lead body within the needle or introducer, then deploy upon removal from the needle or introducer. In some embodiments, the needle or introducer defines one or more cutouts suitable for housing the lead anchoring units during implantation of the lead.

The lead anchoring units include anchoring elements that, upon implantation, lodge against patient tissue and resist, prevent, or reduce rotational, lateral, or axial (or any combination thereof) migration of the lead relative to the patient. The lead anchoring units can be particularly useful for leads for sacral nerve stimulation, spinal cord stimulation, peripheral nerve stimulation, stimulation of cardiac tissue or the stimulation of other patient tissue and organs.

In the case where directional stimulation is performed, prevention of, or resistance to, rotational movement of an implanted lead may be especially important. Conventional stimulation systems may exclusively utilize ring electrodes during stimulation. In which case, rotation of the lead during stimulation may not be of particular importance since stimulation levels may be uniform around the circumference of the lead. In the case of directional stimulation, however, stimulation is often nonuniform around the circumference of the lead and can be radially directed to stimulate tissue in a particular direction and avoid stimulation other tissue around the lead. If the lead were to rotate, however, this directional stimulation may not be directed toward the desired tissue (or may be only partially directed to the desired tissue) which may result in less or no stimulation of the desired tissue. Therefore, resisting or preventing rotation of leads utilizing segmented electrodes is desirable and useful. When the lead anchoring unit is disposed in proximity to the electrodes, particularly the segmented electrodes, and implanted into the patient the lead anchoring unit is configured and arranged for resisting or preventing rotation of the electrodes (e.g., the segmented electrodes) relative to patient tissue.

In the case of sacral stimulation, the implanted lead may be at a higher risk of migrating than with other stimulation locations along the spinal cord (or deep brain stimulation) due to patient movement. Thus, anchoring the lead to maintain positioning of the lead within the sacral region may be especially important. Additionally, with sacral stimulation, the proximity of muscle tissue makes pinpoint accuracy of stimulation important, since it is generally undesired to stimulate untargeted muscle tissue during therapy.

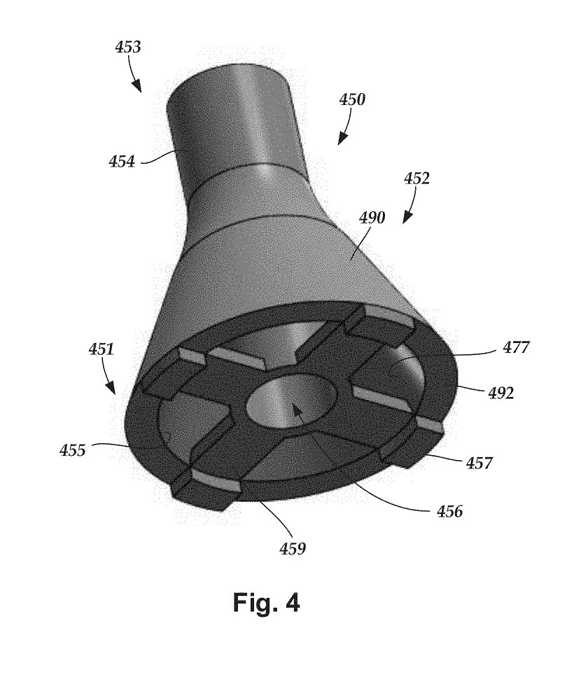

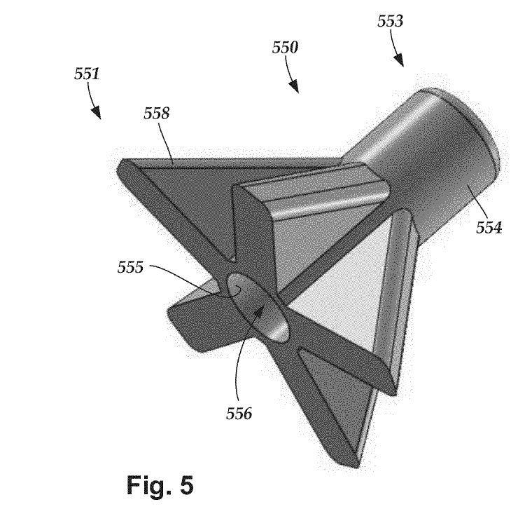

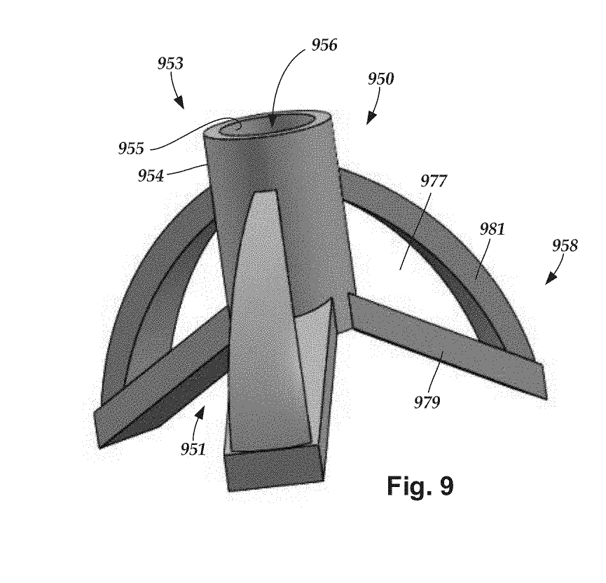

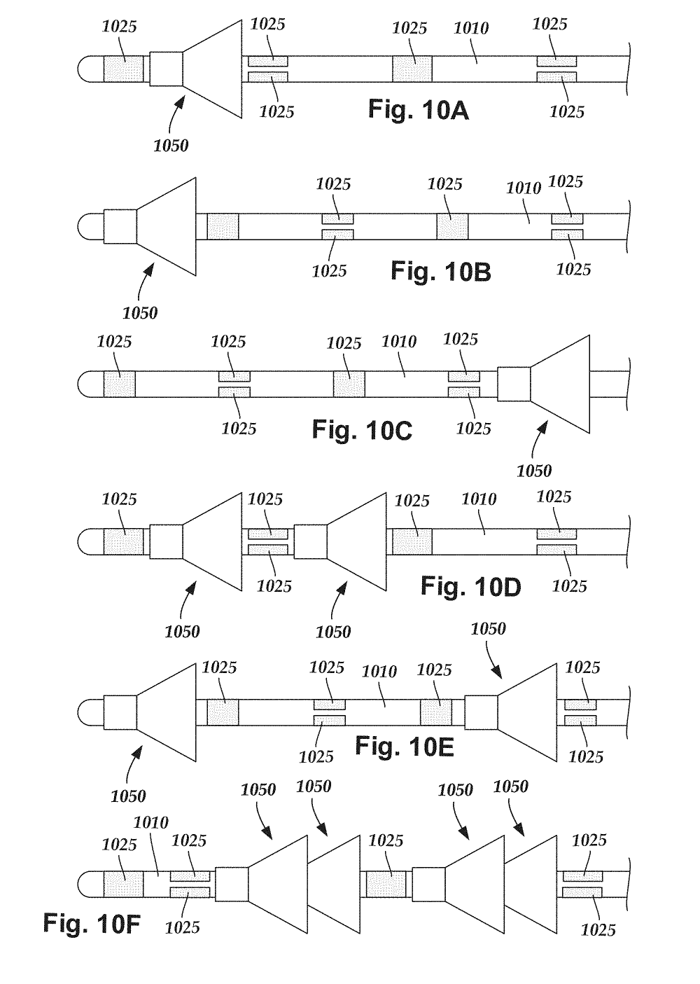

FIGS. 3-10F show several different embodiments of lead anchoring units suitable for mounting along stimulation leads and using in combination with directional stimulation. FIGS. 3 and 4 show several different embodiments of lead anchoring units utilizing a cone, and FIGS. 5-8 show several different embodiments of lead anchoring units utilizing fins. FIG. 9 shows one embodiments of a lead anchoring unit utilizing a combination of cones and fins. FIGS. 10A-10A show several different embodiments of lead anchoring units mounted to a distal portion of a stimulation lead.

The lead anchoring unit described in FIGS. 3-10F facilitate anchoring of the lead body to surrounding tissue when implanted in a patient's body, thereby reducing resisting, or even preventing, rotational movement, lateral movement, axial movement, or some combination thereof, of the portion of the lead in proximity to the lead anchoring unit. When the lead anchoring unit is disposed in proximity to the electrodes, particularly the segmented electrodes, the lead anchoring unit is configured and arranged for resisting or preventing movement (e.g., rotational, lateral, rotational, or combinations thereof) of the electrodes (e.g., the segmented electrodes) relative to patient tissue.

The anchoring units may be configured to facilitate deployment through a needle or introducer. In at least some embodiments, the anchoring units are sufficiently pliable so that it can be compressed within an introducer during implantation. When the introducer is removed, the anchoring units may then expand to anchor the lead body 110 to the tissue. In some embodiments, the needle or introducer defines one or more cutouts defined along a distal end of the needle or introducer for accommodating the anchoring unit.

The anchoring units will be described herein as anchoring a lead within a patient, but it will be understood that the anchoring units can also anchor a lead extension within a patient. Additionally, or alternatively, the anchoring units can anchor a control module of a microstimulator, or a lead of a microstimulator, within a patient.

FIG. 3 is a schematic perspective view of one embodiment of a lead anchoring unit 350 suitable for disposing along a lead body (e.g., a distal portion of the lead body 110 as shown in FIGS. 2A-2D). The anchoring unit 350 includes a lead attachment element 354 having a tube-shaped (e.g., cylindrical) configuration. As shown, the lead attachment element 354 has a proximal end 351, a distal end 353, and a central lumen 356 extending between the two ends 351, 353. The central lumen 356 may be referred to as an “attachment lumen 356”. The attachment lumen 356 is employed to receive at least a portion of the lead body of a lead. In at least some embodiments, the lead attachment element 354 has a circular cross-section. However, the lead attachment element 354 can be formed of any other suitable shape, including shapes having an elliptical, rectangular, polygonal, irregular, or any other suitable lateral cross-section. The lead attachment element 354 can have a uniform lateral cross-section along its entire length or a varying lateral cross-section along its length. In at least some embodiments, the cross-section and dimensions of the lead attachment element 354 are dictated by the configuration of the lead body. In at least some embodiments, the outer diameter of the lead body is slightly larger than the diameter of the attachment lumen 356 so that the lead attachment element fits snuggly on the lead body.

The anchoring unit 350 includes at least one anchoring element 352 coupled to the lead attachment element 354. Although the coupling may occur anywhere along the lead attachment element 354, in the illustrated embodiment, the anchoring element is coupled to the lead attachment element along an intermediate portion of the lead attachment element 354 between the two ends 351, 353. In at least some embodiments, the anchoring element is coupled to the lead attachment element exclusively along an intermediate portion of the lead attachment element 354 and does not physically contact the lead attachment element at either of the two ends 351, 353. In at least some embodiments, the anchoring element does not physically contact the lead attachment element at the distal end 453. In alternate embodiments, the anchoring element does not physically contact the lead attachment element at the proximal end 451.

In the illustrated embodiment, the anchoring element 352 includes a cone 390 that extends over the lead attachment element 354. The cone extends from a first end that attaches to the lead attachment element to a second end (i.e., a lip 392) that is positioned away from the lead attachment element. In at least some embodiments, the cone 390 is longer than the lead attachment element 354 so that the cone 390 extends over, and beyond, the lead attachment element 354. In other embodiments, the cone 390 may be shorter than the lead attachment element and only extend over a portion the lead attachment element.

The anchoring element 352 may include one or more shapes or features to promote interaction with patient tissue to anchor the lead to the tissue. For example, in at least some embodiments, the anchoring unit 350 includes one or more feet 357 extending from the anchoring unit 350 around the lip 392 of the cone. The one or more feet may facilitate a resistance to, reduction of, or prevention of rotation of the anchoring unit 450. The anchoring unit can include any suitable number of feet including one, two, three, four, five, six, seven, eight, nine ten, twelve, fourteen, sixteen, eighteen, twenty, or more feet. When multiple feet are included, the feet can be equally or unequally spaced-apart from one another around the perimeter of the cone, or some combination of both. In FIG. 3, the anchoring unit 350 is shown having four feet 357 equally-spaced apart from one another around the lip 392 of the cone.

In at least some embodiments, an interior surface 355 of the lead attachment element 354 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 350 may form a friction fit with the lead body to hold the anchoring unit in place. Additionally, and alternatively, an adhesive, such as a silicone adhesive, may be employed to bond the anchoring unit 350 to the lead body. In at least some embodiments, the anchoring unit 350 is molded, bonded, and/or RF welded to the lead body. In at least some embodiments, the anchoring unit 350 is permanently attached to the lead body or molded with the lead body. In at least some embodiments, the anchoring unit 350 is placed or positioned on the lead body after manufacture.

The anchoring unit 350 may be formed of any suitable material, such as any suitable biocompatible material including, but not limited to, metals, polymers, alloys, or the like. In at least some embodiments, the anchoring unit 350 is formed of silicone, polyurethane, or the like. In some embodiments, the material that is used has a stiffness that is sufficient to anchor the lead body to the surrounding tissue, while also having sufficient flexibility to reduce, or in some cases avoid, damage or injury to the tissue or to facilitate delivery of the lead with the anchoring unit(s) through an introducer.

FIG. 4 illustrates a second embodiment of a lead anchoring unit 450 suitable for disposing along a distal portion of a lead body. The anchoring unit 450 includes a lead attachment element 454 having a tube-shaped (e.g., cylindrical) configuration. As shown, the lead attachment element 454 has a proximal end 451, a distal end 453, and a central lumen 456 extending between the two ends 451, 453. The central lumen 456 may be referred to as an “attachment lumen 456”. The attachment lumen 456 is employed to receive at least a portion of the lead body of a lead. In at least some embodiments, the lead attachment element 454 has a circular cross-section. However, the lead attachment element 454 can be formed of any other suitable shape, including shapes having an elliptical, rectangular, polygonal, irregular, or any other suitable lateral cross-section. The lead attachment element 454 can have a uniform lateral cross-section along its entire length or a varying lateral cross-section along its length. In at least some embodiments, the cross-section and dimensions of the lead attachment element 454 are dictated by the configuration of the lead body. In at least some embodiments, the outer diameter of the lead body is slightly larger than the diameter of the attachment lumen 456 so that the lead attachment element fits snuggly on the lead body.

The anchoring unit 450 includes at least one anchoring element 452 coupled to the lead attachment element 454. Although the coupling may occur anywhere along the lead attachment element 454, in the illustrated embodiment, the anchoring element is coupled to the lead attachment element at an intermediate portion of the lead attachment element 454 between the two ends 451, 453. In at least some embodiments, the anchoring element does not physically contact the lead attachment element at the distal end 453. In alternate embodiments, the anchoring element does not physically contact the lead attachment element at the proximal end 451.

In the illustrated embodiment, the anchoring element 452 includes a cone 490 that extends over the lead attachment element 454. In at least some embodiments, the cone is longer than the lead attachment element 454 so that the cone extends over, and beyond, the lead attachment element 454. In other embodiments, the cone may be shorter than the lead attachment element and only extend over a portion the lead attachment element.

The anchoring element 452 may have any other suitable shape or feature to interact with patient tissue to anchor the lead to the tissue. For example, in at least some embodiments, the anchoring element 452 includes one or more feet extending from a lip 492 of the cone, as described above with reference to FIG. 3.

In at least some embodiments, the anchoring element 452 includes one or more support members 459 extending radially-outward from the lead attachment unit 454 from a position that is offset along the longitudinal length of the lead attachment element from where the cone 490 attaches to the lead attachment element. The one or more support members 459 attach to the cone 490 at, or in proximity to, the lip 492. The one or more support members 459 may facilitate maintaining the shape of the cone while implanted. Additionally, the one or more support members 459 may promote tissue ingrowth to facilitate anchoring. The anchoring unit can include any suitable number of support members 459 including one, two, three, four, five, six, seven, eight, nine ten, twelve, fourteen, sixteen, eighteen, twenty, or more support elements.

The support members 459 and the cone 490 form apertures, such as aperture 477 that may also be useful for enabling the anchoring unit 450 to be sutured to patient tissue. Any suitable type of suture may be used including, for example, dissolvable sutures. Suturing may be useful, in some cases, for promoting retention of the anchoring units upon implantation at least until enough tissue attaches to the anchoring units to hold the anchoring units in position without aid.

When multiple support elements are included, the support elements can be equally spaced-apart from one another, unequally spaced-apart from one another, or some combination of both. In FIG. 4, the anchoring unit 450 is shown having four support members 459 equally-spaced apart from one another around the lip 492 of the cone. In at least some embodiments, at least one of the support members 459 extends outwardly from the lead attachment unit 454 at a 90-degree angle.

In at least some embodiments, an interior surface 455 of the lead attachment element 454 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 450 may form a friction fit with the lead body to hold the anchoring unit in place. Additionally, and alternatively, an adhesive, such as a silicone adhesive, may be employed to bond the anchoring unit 450 to the lead body. In at least some embodiments, the anchoring unit 450 is molded, bonded, and/or RF welded to the lead body. In at least some embodiments, the anchoring unit 450 is permanently attached to the lead body or molded with the lead body. In at least some embodiments, the anchoring unit 450 is placed or positioned on the lead body after manufacture.

The anchoring unit 450 may be formed of any suitable material, such as any suitable biocompatible material including, but not limited to, metals, polymers, alloys, or the like. In at least some embodiments, the anchoring unit 450 is formed of silicone, polyurethane, or the like. In some embodiments, the material that is used has a stiffness that is sufficient to anchor the lead body to the surrounding tissue, while also having sufficient flexibility to reduce, or in some cases avoid, damage or injury to the tissue or to facilitate delivery of the lead with the anchoring unit(s) through an introducer.

FIG. 5 illustrates a third embodiment of a lead anchoring unit 550 that includes a lead attachment element 554 and at least one anchoring element 558. The lead attachment element 554 has a tube-shaped (e.g., cylindrical) configuration, and includes a proximal end 551, a distal end 553, and a central lumen 556 extending therebetween. The central lumen may also be referred to as “attachment lumen 556”. In at least some embodiments, the lead attachment element 554 has a circular lateral cross-section. However, the lead attachment element 554 can be formed of any other suitable shape, including shapes having an elliptical, rectangular, polygonal, irregular, or any other suitable cross-section. The lead attachment element 554 can have a uniform lateral cross-section along its entire length or a varying lateral cross-section along its length. In at least some embodiments, the cross-section and dimensions of the lead attachment element 554 are dictated by the configuration of the lead body. In at least some embodiments, the outer diameter of the lead body is slightly larger than the diameter of the attachment lumen 556.

In at least some embodiments, an interior surface 555 of the lead attachment element 554 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 550 may form a friction fit with the lead body to hold the anchoring unit in place. Additionally, and alternatively, an adhesive, such as a silicone adhesive, may be employed to bond the anchoring unit 550 to the lead body. In at least some embodiments, the anchoring unit 550 is molded, bonded, and/or RF welded to the lead body. In at least some embodiments, the anchoring unit 550 is permanently attached to the lead body or molded with the lead body. In at least some embodiments, the anchoring unit 550 is placed or positioned on the lead body after manufacture.

The anchoring element(s) 558 are disposed around the lead attachment element 554 and extend away from the lead attachment element 554. In the illustrated embodiment, the anchoring elements 658 are fins. Any number of fins (or other attachment elements) can be used. The embodiment shown in FIG. 5 includes five fins 558 disposed about the circumference of the lead attachment element 554.

The fins 558 shown in FIG. 5 have a triangular-shaped configuration, but it will be recognized that the fins 558 can have any suitable shape including, but not limited to, trapezoidal, rectangular, irregular, and the like. Any suitable number of fins may be disposed about the circumference of the lead attachment element 554 including, but not limited to, two, three, four, five, six, seven, eight, or more fins. The fins can be spaced apart uniformly or non-uniformly around the circumference of the lead attachment element 554. In some embodiments, the fins 558 form an angle of ninety degrees with the lead attachment element, as illustrated in FIG. 5, but it will be recognized that the fins could extend at a different angle from the lead attachment element (for example, an angle in the range from 30 to 85 degrees or 30 to 90 degrees).

The fins 558 are shown in FIG. 5 as being flush with the proximal end 551 of the anchoring unit 550 and extending along a partial length of the lead attachment element 554, while being disposed about the circumference of the lead attachment element 554. However, in some other embodiments the fins extend along an entire length of the lead attachment element 554. In at least some embodiments, the anchoring elements do not physically contact the lead attachment element at the distal end 553.

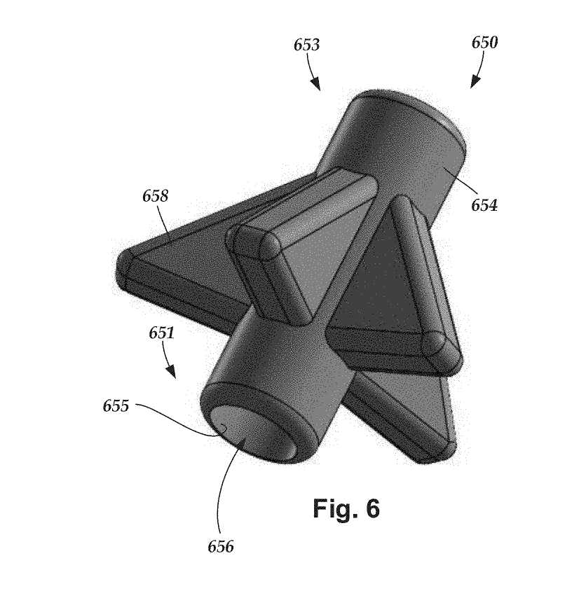

FIG. 6 illustrates a fourth embodiment of a lead anchoring unit 650 that includes a lead attachment element 654 and at least one anchoring element 658. The lead attachment element 654 has a tube-shaped (e.g., cylindrical) configuration, and includes a proximal end 651, a distal end 653, and a central lumen 656 extending therebetween. The central lumen may also be referred to as “attachment lumen 656”. In at least some embodiments, the lead attachment element 654 has a circular lateral cross-section. However, the lead attachment element 654 can be formed of any other suitable shape, including shapes having an elliptical, rectangular, polygonal, irregular, or any other suitable cross-section. The lead attachment element 554 can have a uniform lateral cross-section along its entire length or a varying lateral cross-section along its length. In at least some embodiments, the cross-section and dimensions of the lead attachment element 654 are dictated by the configuration of the lead body. In at least some embodiments, the outer diameter of the lead body is slightly larger than the diameter of the attachment lumen 656.

In at least some embodiments, an interior surface 655 of the lead attachment element 654 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body is generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 650 may form a friction fit with the lead body to hold the anchoring unit in place. Additionally, and alternatively, an adhesive, such as a silicone adhesive, may be employed to bond the anchoring unit 650 to the lead body. In at least some embodiments, the anchoring unit 650 is molded, bonded, and/or RF welded to the lead body. In at least some embodiments, the anchoring unit 650 is permanently attached to the lead body or molded with the lead body. In at least some embodiments, the anchoring unit 650 is placed or positioned on the lead body after manufacture.

The anchoring element(s) 658 are disposed around the lead attachment element 654 and extend away from the lead attachment element 654. In the illustrated embodiment, the anchoring elements 658 are fins. Any number of fins (or other attachment elements) can be used. The embodiment shown in FIG. 6 includes five fins 658 disposed about the circumference of the lead attachment element 654. The fins 658 shown in FIG. 6 have a triangular-shaped configuration, but it will be recognized that the fins 658 can have any suitable shape including, but not limited to, trapezoidal, rectangular, irregular, and the like. Any suitable number of fins may be disposed about the circumference of the lead attachment element 654 including, but not limited to, two, three, four, five, six, seven, eight, or more fins. The fins can be spaced apart uniformly or non-uniformly around the circumference of the lead attachment element 654. In some embodiments, the fins 658 form an angle of ninety degrees with the lead attachment element as illustrated in FIG. 6, but it will be recognized that the fins could extend at a different angle from the lead attachment element (for example, an angle in the range from 30 to 85 degrees or 30 to 90 degrees).

The fins 658 are shown in FIG. 6 as extending along a partial length of the lead attachment element 654 and being disposed entirely along an intermediate portion of the lead attachment element 654 such that the fins 658 do not extend to either the proximal end 651 or the distal end 653 of the lead attachment element 654. In at least some embodiments, the fins are coupled to the lead attachment element exclusively along an intermediate portion of the lead attachment element 654 and do not physically contact the lead attachment element at either of the two ends 351, 353. In at least some embodiments, the fins do not physically contact the lead attachment element at the distal end 453. In alternate embodiments, the fins do not physically contact the lead attachment element at the proximal end 451.

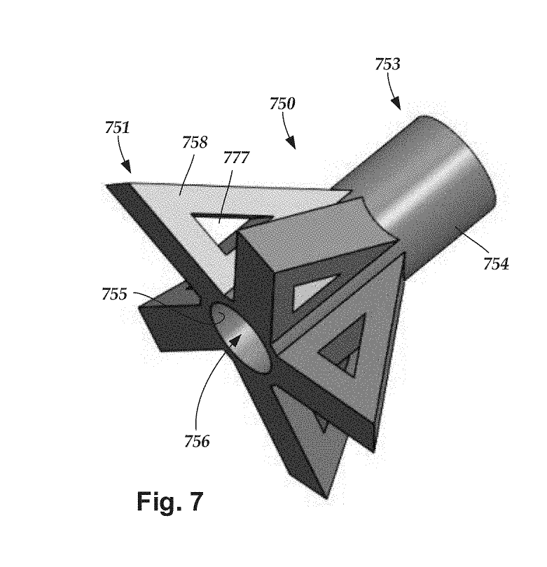

FIG. 7 illustrates a fifth embodiment of a lead anchoring unit 750 that includes a lead attachment element 754 and at least one anchoring element 758. The lead attachment element 754 has a tube-shaped (e.g., cylindrical) configuration, and includes a proximal end 751, a distal end 753, and a central lumen 756 extending therebetween. The central lumen may also be referred to as “attachment lumen 756”. In at least some embodiments, the lead attachment element 754 has a circular lateral cross-section. However, the lead attachment element 754 can be formed of any other suitable shape, including shapes having an elliptical, rectangular, polygonal, irregular, or any other suitable cross-section. The lead attachment element 754 can have a uniform lateral cross-section along its entire length or a varying lateral cross-section along its length. In at least some embodiments, the cross-section and dimensions of the lead attachment element 754 are dictated by the configuration of the lead body. In at least some embodiments, the outer diameter of the lead body is slightly larger than the diameter of the attachment lumen 756.

In at least some embodiments, an interior surface 755 of the lead attachment element 754 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 750 may form a friction fit with the lead body to hold the anchoring unit in place. Additionally, and alternatively, an adhesive, such as a silicone adhesive, may be employed to bond the anchoring unit 750 to the lead body. In at least some embodiments, the anchoring unit 750 is molded, bonded, and/or RF welded to the lead body. In at least some embodiments, the anchoring unit 750 is permanently attached to the lead body or molded with the lead body. In at least some embodiments, the anchoring unit 750 is placed or positioned on the lead body after manufacture.

The anchoring element(s) 758 are disposed around the lead attachment element 754 and extend away from the lead attachment element 754. In the illustrated embodiment, the anchoring elements 758 are fins. Any number of fins (or other attachment elements) can be used. The embodiment shown in FIG. 7 includes five fins 758 disposed about the circumference of the lead attachment element 754. The fins 758 shown in FIG. 7 have a triangular-shaped configuration, but it will be recognized that the fins 758 can have any suitable shape including, but not limited to, trapezoidal, rectangular, irregular, and the like. Any suitable number of fins may be disposed about the circumference of the lead attachment element 754 including, but not limited to, two, three, four, five, six, seven, eight, or more fins. The fins can be spaced apart uniformly or non-uniformly around the circumference of the lead attachment element 754. In some embodiments, the fins 758 form an angle of ninety degrees with the lead attachment element as illustrated in FIG. 7, but it will be recognized that the fins could extend at a different angle from the lead attachment element (for example, an angle in the range from 30 to 85 degrees or 30 to 90 degrees).

As with the fins 558 of FIG. 5, the fins 758 of FIG. 7 are shown as being flush with the proximal end 751 of the anchoring unit 750 and extending along a partial length of the lead attachment element 754, while being disposed about the circumference of the lead attachment element 754. In at least some embodiments, the anchoring elements do not physically contact the lead attachment element at the distal end 753.

The fins 758 of the anchoring unit 750 shown in FIG. 7 include cutouts 777 defined within the fins 758. The cutouts 777 may be useful for facilitating tissue ingrowth which, in turn, may facilitate anchoring of the anchoring units at a target implantation location. The cutouts 777 may also be useful for enabling the anchoring unit 750 to be sutured to patient tissue. Any suitable type of suture may be used including, for example, dissolvable sutures. Suturing may be useful, in some cases, for promoting retention of the anchoring units upon implantation at least until enough tissue attaches to the anchoring units to hold the anchoring units in position without aid.

The embodiment shown in FIG. 7 includes a cutout 777 defined along each of the fins 758, but it will be recognized that the cutouts 777 can be defined along each of the fins 758, or only along a subset of the fins 758. The embodiment shown in FIG. 7 has cutouts 777 that are the same shape as the fins 758 upon which the cutouts 777 are defined, but it will be recognized that the fins 758 can have any suitable shape including, but not limited to, round, oval, capsule-shaped, trapezoidal, rectangular, irregular, and the like. Any suitable number of cutouts 777 may be defined in one or more of the fins 758 including, but not limited to, two, three, four, five, six, seven, eight, or more cutouts 777.

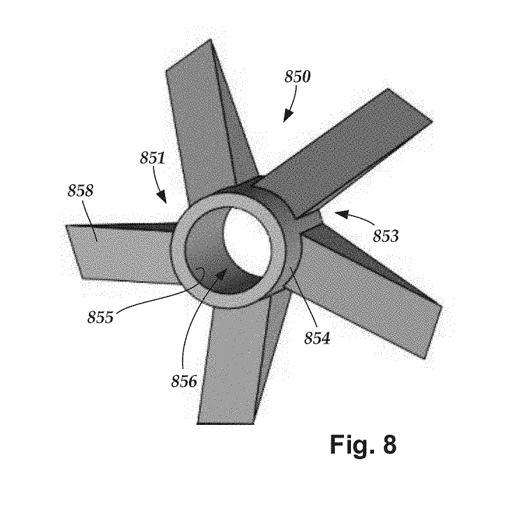

FIG. 8 illustrates a sixth embodiment of a lead anchoring unit 850 that includes a lead attachment element 854 and at least one anchoring element 858. The lead attachment element 854 has a tube-shaped (e.g., cylindrical) configuration, and includes a proximal end 851, a distal end 853, and a central lumen 856 extending therebetween. The central lumen may also be referred to as “attachment lumen 856”. In at least some embodiments, the lead attachment element 854 has a circular lateral cross-section. However, the lead attachment element 854 can be formed of any other suitable shape, including shapes having an elliptical, rectangular, polygonal, irregular, or any other suitable cross-section. The lead attachment element 854 can have a uniform lateral cross-section along its entire length or a varying lateral cross-section along its length. In at least some embodiments, the cross-section and dimensions of the lead attachment element 854 are dictated by the configuration of the lead body. In at least some embodiments, the outer diameter of the lead body is slightly larger than the diameter of the attachment lumen 856.

In at least some embodiments, an interior surface 855 of the lead attachment element 854 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 850 may form a friction fit with the lead body to hold the anchoring unit in place. Additionally, and alternatively, an adhesive, such as a silicone adhesive, may be employed to bond the anchoring unit 850 to the lead body. In at least some embodiments, the anchoring unit 850 is molded, bonded, and/or RF welded to the lead body. In at least some embodiments, the anchoring unit 850 is permanently attached to the lead body or molded with the lead body. In at least some embodiments, the anchoring unit 850 is placed or positioned on the lead body after manufacture.

Although the anchoring element(s) 858 can be disposed anywhere along a longitudinal length of the lead attachment element 854, in the illustrated embodiment the anchoring elements are shown coupled to the lead attachment element at, or immediately adjacent to, its distal end 853. In at least some embodiments, the anchoring elements 858 are flush with the distal end 853 of the lead attachment element 854. In at least some embodiments, the anchoring elements do not physically contact the lead attachment element at the proximal end 851.

The anchoring elements illustrated in FIG. 8 extend along approximately half of the longitudinal length of the lead attachment element 854, but it will be recognized that that the anchoring elements 858 can extend along any portion of the longitudinal length of the lead attachment element 854.

In the illustrated embodiment, the anchoring elements 858 are fins. Any number of fins (or other attachment elements) can be used. The embodiment shown in FIG. 8 includes five fins 858 disposed about the circumference of the lead attachment element 854. The fins 858 shown in FIG. 8 have a triangular-shaped configuration, but it will be recognized that the fins 858 can have any suitable shape including, but not limited to, trapezoidal, rectangular, irregular, and the like. Any suitable number of fins may be disposed about the circumference of the lead attachment element 854 including, but not limited to, two, three, four, five, six, seven, eight, or more fins. The fins can be spaced apart uniformly or non-uniformly around the circumference of the lead attachment element 854. In some embodiments, the fins 858 form an angle of ninety degrees with the lead attachment element as illustrated in FIG. 8, but it will be recognized that the fins could extend at a different angle from the lead attachment element (for example, an angle in the range from 30 to 85 degrees or 30 to 90 degrees).

The anchoring element(s) 858 are disposed around the lead attachment element 854 and extend away from the lead attachment element 854. The anchoring element(s) 954 can extend away from the lead attachment element 854 any suitable length. In at least some embodiments, the anchoring element(s) 858 extend away from the lead attachment element 854 by an amount that is greater than a diameter of the lead attachment element 854 at its widest point. In at least some embodiments, the anchoring element(s) 958 extend away from the lead attachment element 954 by an amount that is greater than the longitudinal length of the lead attachment element 954. In at least some embodiments, the anchoring element(s) 958 extend away from the lead attachment element 954 by an amount that is greater than twice the longitudinal length of the lead attachment element 954.

FIG. 9 illustrates a seventh embodiment of a lead anchoring unit 950 that includes a lead attachment element 954 and at least one anchoring element 958. The lead attachment element 954 has a tube-shaped (e.g., cylindrical) configuration, and includes a proximal end 951, a distal end 953, and a central lumen 956 extending therebetween. The central lumen may also be referred to as “attachment lumen 956”. In at least some embodiments, the lead attachment element 954 has a circular lateral cross-section. However, the lead attachment element 954 can be formed of any other suitable shape, including shapes having an elliptical, rectangular, polygonal, irregular, or any other suitable cross-section. The lead attachment element 954 can have a uniform lateral cross-section along its entire length or a varying lateral cross-section along its length. In at least some embodiments, the cross-section and dimensions of the lead attachment element 954 are dictated by the configuration of the lead body. In at least some embodiments, the outer diameter of the lead body is slightly larger than the diameter of the attachment lumen 956.

In at least some embodiments, an interior surface 955 of the lead attachment element 954 is patterned to assist in maintaining the position of the lead anchoring unit on the lead. The pattern may be regular or irregular and may include features, such as, but not limited to, surface roughening, cutouts, grooves, regular or irregular shapes, or the like. In at least some embodiments, an outer surface of the lead body 110 is patterned. The pattern may be regular or irregular and may include features, such as, but not limited to, surface roughening, cutouts, grooves, regular or irregular shapes, or the like. In some embodiments, both the interior surface of the lead attachment element and the outer surface of the lead body are patterned. The patterning of the lead attachment element and the lead body may be complementary. In at least some embodiments, the pattern on the interior surface of the lead attachment element and the pattern on the exterior surface of the lead body are generated so that the two patterns interlock with each other.

The patterning described above may be formed using any suitable method, including, but not limited to, ablation (e.g., RF or laser ablation), grinding, knurling, chemical etching, or the like. The patterning may be made on the spacers (i.e., between adjacent or consecutive electrodes) of the lead body 110.

The anchoring unit 950 may form a friction fit with the lead body to hold the anchoring unit in place. Additionally, and alternatively, an adhesive, such as a silicone adhesive, may be employed to bond the anchoring unit 950 to the lead body. In at least some embodiments, the anchoring unit 950 is molded, bonded, and/or RF welded to the lead body. In at least some embodiments, the anchoring unit 950 is permanently attached to the lead body or molded with the lead body. In at least some embodiments, the anchoring unit 950 is placed or positioned on the lead body after manufacture.

In the illustrated embodiment, the anchoring elements 958 include a combination of fins and a partial cone formed from a plurality of arced members. Each anchoring element 958 shown in FIG. 9 includes a fin member 979 and an arced member 981 each attached to the lead attachment element 954 and extending outwardly therefrom and coupling to one another so as to form an aperture 977 therebetween. The apertures 977 may be useful for facilitating tissue ingrowth which, in turn, may facilitate anchoring of the anchoring units in a target implantation location. The apertures 977 may also be useful for enabling the anchoring unit 850 to be sutured to patient tissue, as described above with reference to FIG. 7. Additionally, the arc of the arced members 981 may enable the arced member 981 to function as leaf springs to aid in maintaining the shape angle of the fin members 979 relative to the lead attachment element 954 (see e.g., support members 459 of FIG. 4).

Although the anchoring element(s) 958 can be disposed anywhere along a longitudinal length of the lead attachment element 954, in the illustrated embodiment, the anchoring elements are shown coupled to the lead attachment element with the fin member 979 coupled at, or immediately adjacent to, the proximal end 951 of the lead attachment element 954, while the arced member 981 is coupled to an intermediate portion of the lead attachment element 954. In at least some embodiments, fin members 979 are flush with the proximal end 953 of the lead attachment element 954. In at least some embodiments, the anchoring elements do not physically contact the lead attachment element at the distal end 953.

As mentioned above, the arced members 981 may enable the arced member 981 to function as leaf springs via an arced shape. In at least some embodiments, the cone member 981 is tapered such that the width of the cone member 981 increases as the cone member 981 extends away from the lead attachment element 954. The arcing and/or tapering of the arced members 981 can be adjusted to provide the desired rigidity of the fin member 979.

In some embodiments, the anchoring elements 958 form an angle of approximately 60 degrees with the lead attachment element, as illustrated in FIG. 9, but it will be recognized that the anchoring elements could extend at a different angle from the lead attachment element (for example, an angle in the range from 30 to 85 degrees). In at least some embodiments, the anchoring elements 958 extend proximal to proximal end of the lead attachment element 954. The arcing and/or tapering of the arced members 981 can be adjusted to provide the desired angling of the fin members 979.

Any number of anchoring elements 958 can be used. The embodiment shown in FIG. 9 includes three anchoring elements 958 disposed about the circumference of the lead attachment element 954. Any suitable number of anchoring elements 958 may be disposed about the circumference of the lead attachment element 954 including, but not limited to, two, three, four, five, six, seven, eight, or more anchoring elements 958. The anchoring elements 958 can be spaced apart uniformly or non-uniformly around the circumference of the lead attachment element 954.

FIGS. 10A-10F are schematic side views of one embodiment of a distal portion of a lead body 1010, with one or more lead anchoring units 1050 disposed thereon. The distal portion of the lead body 1010 includes multiple electrodes 1025. Eight electrodes 1025 are shown in the illustrated embodiments; however, any suitable number of electrodes 1025 can be provided in any suitable arrangement, including but not limited to two, four, sixteen, or more electrodes. Examples of leads are described above with respect to FIGS. 1-2D and the references cited herein.

One or more anchoring units 1050 can be mounted on the lead body 1010. In the embodiment illustrated in FIG. 10A, the anchoring unit 1050 is mounted between adjacent electrodes (e.g., ring electrodes or sets of segmented electrodes) 1025. In the embodiment illustrated in FIG. 10B, the anchoring unit 1050 is mounted distally from the electrodes 1025. In the embodiment illustrated in FIG. 10C, the anchoring unit 1050 is mounted proximally from the electrodes 1025. In the case of sacral nerve stimulation, it may be an advantage to position at least one of the anchoring units proximal to the electrodes to reduce the risk of migration of the lead through the foramen.

In the embodiment illustrated in FIG. 10D, two anchoring units 1050 are mounted between the adjacent electrodes (e.g., ring electrodes or sets of segmented electrodes) 1025, with each of the anchoring units 1050 mounted between different adjacent electrodes. In the embodiment illustrated in FIG. 10E, one anchoring unit 1050 is disposed distally from the electrodes 1025 and one anchoring unit 1050 is disposed between the adjacent electrodes (e.g., ring electrodes or sets of segmented electrodes) 1025. In the embodiment illustrated in FIG. 10F, two sets of two anchoring units 1050 each are mounted between the electrodes (e.g., ring electrodes or sets of segmented electrodes) 1025, with each set of the anchoring units 1050 mounted between a different grouping of adjacent electrodes. It will be understood that other embodiments may include any suitable combination of one or more anchoring units being mounted proximal to, distal to, between two or more electrodes (e.g., ring electrodes or sets of segmented electrodes), or some combination thereof.

The anchoring units 1050 may be any of the anchoring units describe above, including the anchoring units 350, 450, 550, 650, 750, 850, and 950 of FIGS. 3, 4, 5, 6, 7, 8, and 9, respectively. Any suitable number of anchoring units 1050 may be used, including two, three, four, five, six, seven, eight, nine, ten, or more anchoring units. When used in combinations of two anchoring units, the anchoring units may be all of the same type or of some combination of different types (cone-cone, cone-fin, fin-cone, or fin-fin). Additionally, the combination anchoring unit 950 can be used in combination with fin-type anchoring units, cone-type anchoring units, or both.

In some situations (see e.g., FIG. 10F), it may be advantageous to position several anchoring units in close proximity to one another (e.g., abutting one another axially) along the lead body. Such positioning may improve anchoring. Positioning an anchoring unit with anchoring element(s) disposed along an intermediate portion of the lead attachment element (e.g., anchoring unit 650) axially adjacent and proximal (along a longitudinal length of the lead) to an anchoring unit having anchoring element(s) disposed along the proximal end of the lead attachment element (e.g., anchoring unit 550) may enable the anchoring elements of the two anchoring units to overlap with one another. Such positioning may further enhance anchoring.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An implantable electrical stimulation lead comprising:
a lead body having a distal portion, a proximal portion, a perimeter, and a longitudinal length;
a plurality of electrodes disposed along the distal portion of the lead body, the plurality of electrodes comprising a plurality of segmented electrodes, wherein each of the segmented electrodes extends around no more than 50% of the perimeter of the lead body;
a plurality of terminals disposed along the proximal portion of the lead body;
a plurality of conductors electrically coupling the terminals to the electrodes; and
at least one anchoring unit disposed along the distal portion of the lead body and configured and arranged for resisting rotation of the lead body and electrodes relative to patient tissue after implantation of the lead, the at least one anchoring unit comprising
a lead attachment element defining a central lumen within which a portion of the lead body is received, the lead attachment element having a proximal end, a distal end, a diameter, and a longitudinal length, and
at least one anchoring element attached to the lead attachment element and extending away from the lead attachment element, the at least one anchoring element configured and arranged for physically contacting patient tissue and resisting rotation of the lead body in proximity to the at least one anchoring element.

2. The electrical stimulation lead of claim 1, wherein the at least one anchoring element is attached to the lead attachment element at the proximal end of the lead attachment element.

3. The electrical stimulation lead of claim 1, wherein the at least one anchoring element is attached to the lead attachment element at the distal end of the lead attachment element.

4. The electrical stimulation lead of claim 1, wherein the at least one anchoring element is attached to the lead attachment element exclusively at an intermediate portion of the lead attachment element between the proximal end and the distal end.

5. The electrical stimulation lead of claim 1, where the at least one anchoring element comprises a cone having a first end and an opposing second end, the first end attached to the lead attachment element and the second end longitudinally spaced away from the lead attachment element.

6. The electrical stimulation lead of claim 5, wherein the at least one anchoring element comprises at least one foot disposed along the second end of the cone.

7. The electrical stimulation lead of claim 1, where the at least one anchoring element comprises a plurality of fins, each fin attached to, and extending away from, the lead attachment element.

8. The electrical stimulation lead of claim 7, wherein at least one of the plurality of fins defines a cutout.

9. The electrical stimulation lead of claim 1, wherein each of the at least one anchoring elements comprises a first member attached to the lead attachment element at a first location and a second member attached to the lead attachment element at a second location that is discrete from, and proximally-offset from, the first location along the longitudinal length of the lead attachment element.

10. The electrical stimulation lead of claim 9, wherein the first member extends from the lead attachment element and couples to the second member to form at least one aperture bounded, at least in part, by the first member and the second member.

11. The electrical stimulation lead of claim 9, wherein the first member extends along an arced path from the second member to the lead attachment element.

12. The electrical stimulation lead of claim 9, wherein the first member has a width, and wherein the width tapers from the second member to the lead attachment element.

13. The electrical stimulation lead of claim 9, wherein the second member extends from the lead attachment element at the proximal end of the lead attachment element.

14. The electrical stimulation lead of claim 9, wherein the second member extends from the lead attachment element at a 90-degree angle from the lead attachment element.

15. The electrical stimulation lead of claim 1, wherein the at least one anchoring element extends away from the lead attachment element by an amount that is greater than the longitudinal length of the lead attachment element.

16. An electrical stimulation system, comprising:
the electrical stimulation lead of claim 1; and
a control module coupleable to the electrical stimulation lead, the control module comprising
a housing, and
an electronic subassembly disposed in the housing.

17. A method for sacral stimulation to treat a condition, the method comprising:
advancing the distal portion of the electrical stimulation lead of claim 1 to a location within a patient in proximity to at least one nerve root of at least one of the S1, S2, S3, S4, or S5 level of the patient's spinal cord;
anchoring the distal portion of the electrical stimulation lead to patient tissue using the at least one anchoring unit to resist rotational movement of the plurality electrodes of the electrical stimulation lead relative to the at least one nerve root; and
providing electrical signals from a control module coupled to the electrical stimulation lead to stimulate the at least one nerve root using the plurality of electrodes of the electrical stimulation lead.

18. The method of claim 17, wherein anchoring the distal portion of the electrical stimulation lead to patient tissue comprises anchoring the distal portion of the electrical stimulation lead to patient tissue to resist axial movement of the plurality of electrodes of the electrical stimulation lead relative to the at least one nerve root.

19. The method of claim 17, wherein the condition is one or more of overactive bladder, urinary incontinence, bowel incontinence, urinary retention, fecal retention fecal retention, fecal incontinence, intersticial cistytis, pelvic pain, erectile dysfunction, female sexual dysfunction, or pelvic floor tone dysfunctions.

20. An implantable microstimulator comprising:
a control module comprising
a housing having an outer surface, and
an electronic subassembly disposed in the housing;
a plurality of electrodes disposed along the outer surface of the housing and coupled to the electronic subassembly; and
at least one anchoring unit disposed along the outer surface of the control module and configured and arranged for resisting movement of the control module relative to patient tissue after implantation of the microstimulator, the at least one anchoring unit comprising
a lead attachment element defining a central lumen within which a portion of the control module is received, the lead attachment element having a proximal end, a distal end, a diameter, and a longitudinal length, and
at least one anchoring element attached to the lead attachment element and extending away from the lead attachment element, the at least one anchoring element configured and arranged for physically contacting patient tissue and resisting movement of the control module in proximity to the at least one anchoring element.
